(19) United States
(12) Patent Application Publication
(10) Pub. No.: US 2023/0420093 A1
(43) Pub. Date: Dec. 28, 2023

(54) METHODS AND SYSTEMS FOR COLLECTING AND PROCESSING DATA FOR GENERATING PATIENT SUMMARY AND GUIDANCE REPORTS

(71) Applicant: e-Lovu Health, Inc., Los Gatos, CA (US)

(72) Inventors: Santosh Pandipati, Los Gatos, CA (US); Noel M. Pugh, San Francisco, CA (US); Dharanipathy Rangaraj, Austin, TX (US); Sean Malynn, San Jose, CA (US)

(21) Appl. No.: 17/850,951

(22) Filed: Jun. 27, 2022

(51) Int. Cl. G16H 15/00

(52) U.S. Cl. CPC G16H 15/00

(57) ABSTRACT

Methods, systems, and computer programs are presented for processing pregnancy care data for presentation via an information portal. The method includes accessing a plurality of data sources having care data related to a patient. The method includes filtering data from the plurality of data sources. The filtering is configured to identify relevant data to the patient responsive to a request for a guidance report for pregnancy care of the patient via the information portal. The method includes identifying at least one pattern from the relevant data. The method includes generating the guidance report regarding the patient. The guidance report includes relevant data from one or more prior visits with a care provider.

### BACKGROUND

### 1. Field of the Invention

The present embodiments relate to methods and systems for processing data obtained from disparate sources for optimizing accuracy of information used for delivery of care to expecting mothers by health professionals.

### 2. Description of the Related Art

Current pregnancy care consists of sequential visits from soon after conception until delivery, and through six weeks postpartum. These visits are comprised of patient education, assessment of vital signs, ordering of screening tests per American College of Obstetrics and Gynecology (ACOG) guidelines, evaluation of out-of-range clinical parameters, delivery planning, and assessment of postpartum recovery.

Unfortunately, documentation of prenatal care is fragmented across various sources, e.g., electronic health record systems (EHRs), laboratory facilities, hospital EHRs (for patient emergency visits, admissions, deliveries, etc.) etc. For patients consulting with allied health professionals such as nutritionists, diabetes educators, behavioral health therapists, physical therapists, doulas, lactation specialists, etc. there is often a lack of clear communication between these allied health professionals and a mother's primary care team. Consequently, pregnancy care providers may sometimes be unaware of information that may be important to the care being administered.

It is in this context that embodiments arise.

### SUMMARY

Systems, devices, methods, and computer programs are disclosed and relate to the collection of data from multiple data sources, processing the data to assess pregnancy care history and generation of guidance summaries. In one embodiment, the collection of data is automated and accessed from different sources. Some data may be public data, e.g., published care articles, pregnancy care standards, pregnancy care software guides and other data may be private, e.g., a patient mother's remote monitoring data, electronic health records, electronic discussions with a care provider (e.g., text discussions, email discussions, and/or voice and video discussions). In one configuration, the collected data is processed by one or more services of a cloud system in order to filter data.

The cloud system, in one embodiment, is defined by one or more servers and storage. The cloud system may function in one or more data centers to enable efficient remote access by care providers and patients. By way of example, the cloud system will execute one or more programs and processes to enable the collection of data and processing in order to generate guidance summaries. The programs and processes may include code for executing functionality for a care processing system and an information portal, as described in more detail with respect to FIG. 1 below.

In one embodiment, the filtering implements rules to find specific information from the data sources, e.g., data regarding a female of a certain age, a certain weight, a certain health history, a certain demographic, a certain geolocation, certain medical parameters of the patient, and extracted filtered data from the disparate data sources. Once the data has been filtered, the data can be used to populate predefined descriptive metrics or templates of the patient within one or more data structures managed by the care processing system. In one embodiment, the care processing system is configured to receive requests from a care provider or a patient at various times during a pregnancy. The request, in one embodiment, triggers the generation of a guidance report that will include information regarding identified pattens, trends and/or insights. In this configuration, the guidance report can be requested at any time during the pregnancy.

When it is requested, the information used for generating the guidance report will advantageously utilize information of prior visits to the care provider and information obtained from other sources. The other sources are those that are automatically accessed by the care processing system and/or programmed to be accessed by an administrator of the care processing system. Further, in prior visits, the care provider may additionally prescribe care recommendations, treatments, and/or medications. Data regarding the prescriptions provided by a care provider at any time during the pregnancy will also be feedback to the care processing system, and will be at least part of the basis for a future summary guidance report by the same provider or any other next provider. As can be appreciated, as care is administered during pregnancy, the care cannot only be customized for the mother, but will also continually be based on the latest care previously provided, the latest trends detected in other patients, and other predictive analysis.

In one embodiment, the care processing system disclosed herein overcomes many of the problems known to exist with traditional (electronic health record systems) EHRs, which do not act as interactive, analytical, or predictive engines, and as such they do not provide evidence-based anticipatory care guidance for primary care providers and patients based on accumulated patient care data.

In one embodiment, the care processing system transforms pregnancy care data into targeted guidance which provides better prenatal care situational awareness for clinical care teams, patients, allied health professionals and providers. As mentioned above, the care processing system interfaces with multiple pregnancy care data sources. In one embodiment, the care processing system collects relevant data from these sources, identifies key pregnancy care patterns, trends and insights and provides holistic and directed pregnancy care guidance and recommendations. It is then displayed on a computer and/or on a mobile application for viewing by the pregnancy care teams, patients, allied health professionals and providers.

In one embodiment, a method for processing pregnancy care data for presentation via an information portal is provided. The method includes accessing a plurality of data sources having care data related to a patient. The method includes filtering data from the plurality of data sources. The filtering is configured to identify relevant data to the patient responsive to a request for a guidance report for pregnancy care of the patient via the information portal. The method includes identifying at least one pattern from the relevant data. The method includes generating the guidance report regarding the patient. The guidance report includes relevant data from one or more prior visits with a care provider.

In some embodiments, the plurality of data sources include data related or collected from the patient and data related to a patient population having similar care characteristics to the patient, wherein the similar characteristics are at least partially used to assist in identifying said at least one pattern.

In some embodiments, the filtering data further includes using a machine learning processor that is configured to parse collected data from each of the plurality of data sources to identify features for related types of data. The features are processed by classifiers that are specific to the types of data, wherein output of the classifiers are processed to generate a patient model. The patient model is trained over time using feedback from the care provider or the patient.

In some embodiments, the guidance report, when generated, provides metrics recorded in said one or more prior visits. The metrics are present in a descriptive format that is presented in one or more user interfaces. The one or more user interfaces are for display on a user device having access to the care processing system via the information portal.

In some embodiments, the identified at least one pattern from the relevant data is identified using a machine learning processor that extracts feature data from the plurality of data sources and labels features for classification and processing by model. The model is used to identify said at least one pattern or generate an insight to be presented in the guidance report.

In some embodiments, the plurality of data sources includes patient population data and associated feedback learning data to train said model, including data sourced from other online care providers via one or more Application Programming Interfaces (APIs).

In some embodiments, a machine learning model is used to identify said at least one pattern.

In some embodiments, one or more rules engines are used to identify said at least one pattern.

In some embodiments, the rules engines are programmable to identify specific data from said plurality of data sources and apply logic to represent said specific data in a descriptive format that describes an aspect of prior care of the patient.

It should be appreciated that the present embodiments can be implemented in numerous ways, such as a method, an apparatus, a system, a device, or a computer program on a computer readable medium. Several embodiments are described below.

### BRIEF DESCRIPTION OF THE DRAWINGS

The embodiments may best be understood by reference to the following description taken in conjunction with the accompanying drawings.

FIG. 1 illustrates a block diagram associated with a pregnancy care delivery assistant (PCDA) 100, in accordance with one embodiment.

FIG. 2 illustrates an example of the pregnancy care delivery assistant (PCDA), where machine learning is utilized as part of the filtering process, in accordance with one embodiment.

FIG. 3 illustrates an example of the PCDA, where processing done by the machine learning processor utilizes data gathered from the data sources, which include data source, in accordance with one embodiment.

FIG. 4 illustrates an example of a pregnancy timeline, wherein a first trimester, second trimester, and a third trimester is illustrated broadly for discussion purposes, in accordance with one embodiment.

FIGS. 5 and 6 illustrate example data flow configurations when receiving data (e.g., data sources) from different pregnancy care ecosphere partners, in accordance with one embodiment.

FIG. 7 illustrates an example of the multiple sources of pregnancy care data and how collected data is then organized to define the patient's relevant collected data, in accordance with one embodiment.

FIG. 8 illustrates an example where the patient's relevant collected data can be aggregated and made to generate relevant information that can be presented in a guidance report, in accordance with one embodiment.

FIG. 9 illustrates an example of utilizing the types of patterns, trends and insights in order to generate a summary guidance report, in accordance with one embodiment.

FIG. 10 illustrates an example of the visualization that can be created from the data in the summary guidance report, in accordance with one embodiment.

FIG. 11 is a screenshot example showing an individual patient dashboard that is displayed as a result of the PCDA output, in accordance with one embodiment.

FIG. 12 is a screenshot example showing an all-site population dashboard that is displayed as a result of the PCDA output, in accordance with one embodiment.

FIG. 13 is a screenshot example showing the specific site population dashboard that is displayed as a result of the PCDA output, in accordance with one embodiment.

Other aspects will become apparent from the following detailed description, taken in conjunction with the accompanying drawings.

### DETAILED DESCRIPTION

The following embodiments describe systems, devices, methods, and computer programs for collecting and processing data from multiple data sources, processing the data to assess pregnancy care history and generation of guidance summaries. In one embodiment, the collection of data is automated and accessed from different sources using a care processing system that executes one or more processes on one or more servers. In one embodiment, the care processing system provides an information portal that enables access to care providers and mothers during pregnancy and child birth. The information portal is configured to access one or more summary guidance reports and present metrics via one or more user interfaces rendered on a user device (e.g., a computing device having Internet access). The user interface can provide in an easy-to-read dashboard that dynamically renders the summary guidance data upon request.

In one embodiment, the summary guidance data is rendered using information gathered from prior visits of the mother and from public data, e.g., published care articles, pregnancy care standards, medical journals, pregnancy care software guides and other data. For the specific mother, the care processing system will also have access to her remote monitoring data, electronic health records, electronic discussions with a care provider (e.g., text discussions, email discussions, and/or voice and video discussions). In one embodiment, machine learning is used during the filtering of the collected data. Using machine learning, a personalized patient model can be constructed. The personalized patient model can thus be used by the care processing system to improve the identification of patterns in the mother's health, trends and insights. This information, once processed by the care processing system is used by the information portal to present data for the summary reports, dashboard information and predictive data.

As mentioned, the care processing system is programmable to interface with one or more of pregnancy care data sources like hospital electronic health records, remote patient monitoring sources, published pregnancy care standards and articles, SMS texting conversations between care providers and patients, prenatal visit scheduling systems, current billing code standards, etc.

In one embodiment, once these interfaces are established (e.g., programmed), the care processing system can extract pertinent patient data for further prenatal care analysis. To ensure data relevancy, this extraction process will include pregnancy care data content filtering based on permissions, controls, and settings. Some examples of this data, post extraction, are the specific pregnancy care metrics such as the mother's vital signs or weight, lab results, ultrasound data, allied health engagement, care protocol adherence, and high frequency pregnancy care clinical text usage during SMS communication between care teams and their patients. In one embodiment, the care processing system can collect similar data on specified patient populations.

With relevant patient or population data collected, the care processing system will aggregate and mine this data for relevant pregnancy care patterns, trends, and insights using machine learning (ML), artificial intelligence (AI) and other investigational techniques. The care processing system will use the pertinent data and generate information that provides users with precise guidance to assist in achieving high quality pregnancy care outcomes and population metrics. Some example outputs of this prenatal data analysis are identification of out-of-range vital signs since the last prenatal visit along with specific values, weight gain since prior visit, mapped against pregnancy weight gain goals and standards, technical difficulties from remote patient monitoring and outcomes of the technical support, patient visits with allied health professionals, and next ACOG guideline tasks such as gestational diabetes screening, Tdap vaccine administration, or GBS culture.

Examples of pregnancy population patterns, trends and insights include total c-section deliveries per physician on site, allied health services used in a population, average time saved due to remote monitoring and telehealth appointments. Furthermore, predictive analytics will also be applied to make specific recommendations for number and type of interactions with the multitude of allied health pregnancy care providers based on historical and observed outcomes. Using predictive and descriptive data analysis a comprehensive list of insights, patterns, and trends is generated to facilitate creation of a prenatal summary guidance report for the patient and her care team.

In one embodiment, the care processing system will translate and synopsize the patterns, trends, and insights (using AI/ML and other investigational techniques) to create a summary guidance report. This summary guidance report is an overview of key learnings and suggested actions to take. Some examples of this are patient had two out of range high blood pressure readings of 150/110 since the last prenatal visit, lab results and ultrasound measurements are within range, ACOG recommends a diabetes screening test before the next prenatal visit, SMS text indicates high frequency usage of term “urinary incontinence”—recommend patient to seek pelvic floor physical therapy and undergo further evaluation for possible gestational hypertension or preeclampsia.

With the synopsized summary guidance, the care processing system will transfer the data and create visualizations and display a report on a computer and/or within an application. The users of these dashboards are pregnancy care navigators and administrators, pregnancy care teams, patients, allied health professionals and providers, health systems, employers, and insurance payers.

The care processing system is a technology platform that extracts pregnancy care data, filters and analyzes this data (using ML/AI and other investigational techniques) and yields precise and comprehensive guidance for care providers and patients based on accumulated care data. This data collection, transformation and analytics tool removes fragmented pregnancy care data, creates a mechanism to aid clear communication among patient's care teams and produces anticipatory pregnancy care guidance.

In one embodiment, ancillary descriptive and predictive analysis will also include environmental and cost saving considerations associated with pregnancy care. Examples of environmental metrics are carbon emission reduction variables, patient distance from clinic or delivery unit, number of pregnancy care telehealth visits compared to standard all in-person visits, patient's primary vehicle and gas mileage, number of avoided labor and delivery or emergency department visits. Examples of cost savings variables are average cost of gas at time of avoided in-office visit, retained wages, retained PTO, retained childcare costs.

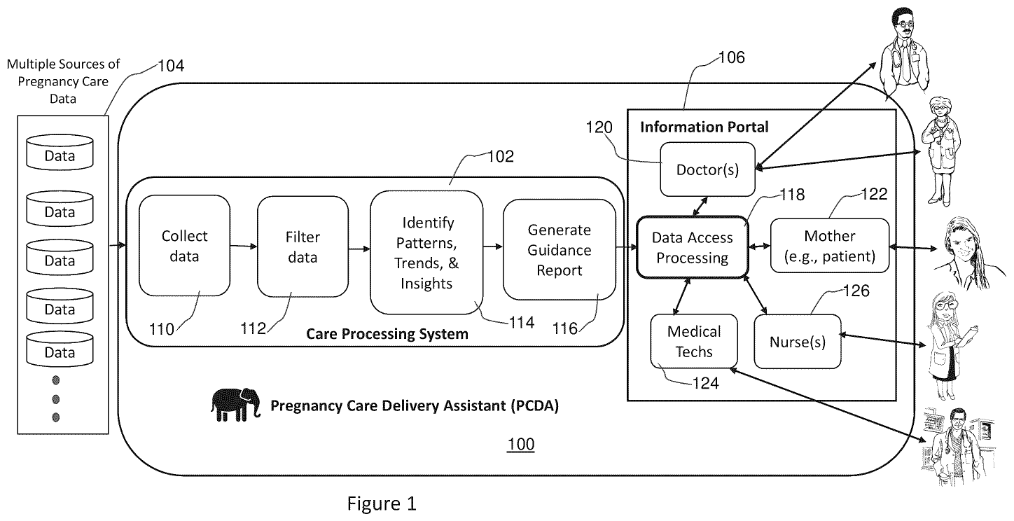

FIG. 1 illustrates a block diagram associated with a pregnancy care delivery assistant (PCDA) 100, in accordance with one embodiment. The PCDA 100, in one embodiment, is part of an online service and network that provides pregnancy care providers with access with accurate and synthesized information to enable efficient care to a mother/patient. The online service, in example embodiment, is part of a product provided by e-Lōvu Health (current assignee). By way of example, the PCDA 100 includes several components, such as a care processing system 102, and an information portal 106. Additionally, multiple data sources 104 are interfaced with the PCDA 100 using various linking protocols that enable access of the multiple and disparate data sources and processing of that data for use by the care processing system 102, in accordance with one embodiment. In configuration, the data sources 104 include data obtained from private entities that collect information related to pregnancy and/or collect information related to a specific patient that is utilizing or can utilize the PCDA 100. By way of example, some data sources can be associated with other care providers that provide electronic care services or in-person care services to a patient or many patients as a service.

This information gathered by other care providers may be stored in their respective databases. In some embodiments, collaboration with these other care providers will require engineered interfaces to allow sharing of sensitive data between databases. In some embodiments, application-programming interfaces (APIs) are established between the providers backend servers to enable sharing of specific data. By way of example, if the data is sensitive, encryption can be provided for the API transfers from the other providers databases to the database or databases of the PCDA 100. In some embodiments, data is more publicly accessible and those data sources can be accessed by programming engines that allow for reading of specific data, parsing of data, and retrieval of data for use by the PCDA 100. In some embodiments, the PCDA 100 can include a user interface that allows a patient or care provider or administrator to link access to other electronic care services. The access will allow the PCDA 100 to ingest specific data that may be relevant to the patient, e.g., prior visits by the patient with such other providers, and/or online visit data of the patient with such one or more other providers.

By way of example, these databases can include pregnancy care software systems, pregnancy care standards, published care articles, and other external or internal data sources. As can be appreciated, the multiple sources of pregnancy care data 104 can be substantial, and the care processing system 102 will collect data 110, in order for additional processing. Furthermore, the pregnancy care data sources 104 can also include data that is specific to a patient/mother, and many other patients and mothers. By way of example, the patient data can include remote patient monitoring data that is collected from one or more online visits. As used herein, online visits relate to care sessions delivered by care professionals to the patient where patient data can be obtained, stored, and updated.

The online/virtual visits can include monitoring of vital signs of the patient, pregnancy metrics, patient feedback, test data feedback, remote electronic monitoring, video data, audio data, text data, and other information that can be collected during the online visit. In some embodiments, the online visit can be performed using a voice telephone call, or a combination of voice and video, and/or a computer conference call that allows for person-to-person communication between the care provider and the patient.

In some embodiments, the patient data can include electronic health records that may relate to the patient, text discussions with the patient, feedback gathered by the care professional related to a visit, and the like. In some embodiments, visits can be in-person, where the care professional examines the patient, provides guidance, obtains pregnancy metrics, schedules appointments, performs care routines, e.g., ultrasounds, lab results, examines lab results, provides medication, reviews medication, provides consultation, and the like. In other embodiments, the visits can be a hybrid model, where some visits are done virtually and some visits are done in person. Information and data gathered from both in-person and online/virtual visits can then be stored in one or more databases of the PCDA 100, and/or obtained from the data sources 104 insubstantial real-time or cached for later access in collected data 110.

In one embodiment, virtual visits can be enabled using a system that qualifies the patient to determine if the patient is eligible to receive virtual (e.g., online, on-phone, video call, etc.) care. Some eligibility processes can include asking the patient to respond to a questionnaire, e.g., via an online application, to assess the patient's current pregnancy status and associated metrics. Some examples include, asking when the patient's last menstrual period occurred. If it is not within e.g., a pre-specified gestational age, then the patient may not be eligible for virtual care. If it is within e.g., a pre-specified gestational age, then data can be gathered, such as dating and viability ultrasound, general physical exam, baseline labs, insurance eligibility, etc. Once this additional information is gathered, then additional logic will determine if the patient is eligible for virtual care or a tele-chat via the app. If the patient is eligible, then additional personal vital data can be collected, either in person or using remote monitoring. These examples are provided to illustrate that some of the visits of the patient can be virtual and some can be in-person. The collected data 110 can therefore be from either visit type or combination of types.

Collected data 110 from the data sources 104 is then processed by a filtering system 112, the filters data to identify specific data relevant to a patient. By way of example, if a care provider is requesting a guidance report for a patient, the filtering of data will access data relevant to the patient from the collected data 110. The filtering of data, and one embodiment, can include utilizing machine learning to identify not only specific data tagged or marked for the patient, but also to make assumptions and predictions of data that may be relevant to the patient. Machine learning can be utilized to analyze big data that is collected from a population of patients that may have similar characteristics to the patient's being examined. This analysis will allow for predictive patterns in the data to be identified, and such information can be presented in a guidance report 116 generated by the care processing system for the patient.

In one embodiment, the identified patterns can also be associated with trends that can identify suggestions to be made in the guidance report for the patient. By way of example, if prior visits generated specific data that indicates a possible issue with the care being provided, the guidance report can provide insights for the doctor 120 or nurse 126 or patient 122 that may be acted upon before the situation becomes urgent. By way of example, the patterns, trends, and insights 114 processed by the care processing system 102 will allow for the generation of the guidance report 116 with information that is not simply stagnant or read directly from a database, but also suggestive of possible treatments, possible issues, and possible preventative care the can be rendered to the patient.

Furthermore, these preventative measures are additionally assisted when the data sources 104 are from other care providers. Other care providers may also be providing guidance to the patient in areas that are complementary to the pregnancy care. For instance, the care can be related to nutrition, behavioral and/or psychological treatment, pregnancy guidance, exercise, and/or pregnancy mentoring by an electronic doula. It should be appreciated that the generated guidance report 116 will be able to provide more comprehensive data regarding the patient when additional data sources are considered.

This is a significant difference from previous systems that operate in isolation from other care providers. By blending and accessing information from multiple care providers that may be providing care to the patient it is possible to generate a guidance report that is more comprehensive, not repetitive, and takes into account previously rendered care to avoid conflicts. Traditionally, these insights are only gathered by highly skilled care providers with many years of experience, knowing that other care providers may have information relevant before care is provided. However, even highly skilled care providers are not able to know all of the previous care rendered by other systems, or even information in all relevant and up-to-date care publications, articles, pregnancy standards, and the like.

Continuing with FIG. 1, an information portal 106 is managed by the PCDA 100, to provide access to the data produced by the care processing system 102, in accordance with one embodiment. Data access processing 118 is utilized as an interface to the care processing system 102. The data access processing 118 is the interface that allows external access in a controlled manner to the PCDA 100 by care providers and patients. As shown, doctors 120, mothers (e.g., patients) 122, medical technicians 124, nurses 126, and other entities requiring access to the guidance information report 116 can be granted access via the data access processing 118. In one embodiment, credentials are utilized to provide access to specific users. The credentials can allow for different levels of access to information depending on the credentialing level. This ensures for privacy concerns related to the information and also enables monitoring of access history, verification of credentials, and granular settings for different types of data and their respective access.

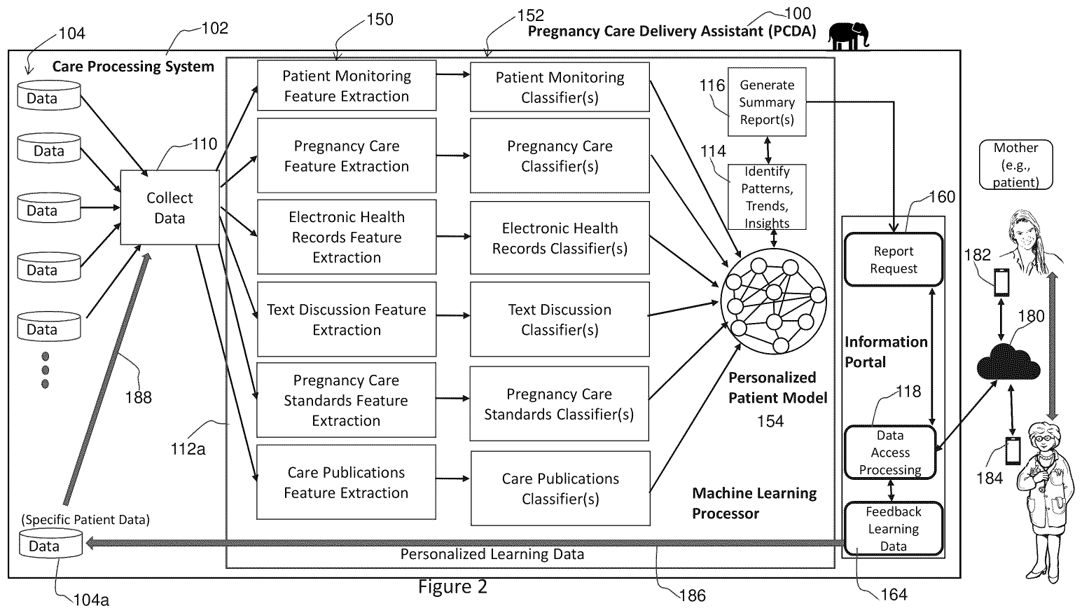

FIG. 2 illustrates an example of the pregnancy care delivery assistant (PCDA) 100, where machine learning is utilized as part of the filtering process, in accordance with one embodiment. As shown, the data sources 104 discussed above can be accessed by the care processing system 102. Further shown is data source 104a that is specific to the patient for which care is being provided. The specific patient data is additionally added to the collected data 110 and stored in the respective entry or entries of a database. Additionally, feedback learning data 164 is generated based on the visit and or consultation with a care provider, which enables personalized learning data 186 to be part of the specific patient data 104a. The feedback learning data 164 can therefore be utilized by the machine learning processor to perform additional feature extraction and classification that is fed to the personalized patient model 154. As shown, the collected data 110 can produce different types of data. Different types of data are shown, by way of example, and not exclusive of any other type of data that can be collected.

The example types of data that can be feature extracted can include patient monitoring data, pregnancy care data, electronic health record data, text discussion data, pregnancy care standards data, care publication data, and the like. These data sources that are collected 110 are then processed by filtering 112a to identify features that are labeled during feature extraction 150. These labeled features are then fed to respective classifiers 152. The classifiers will then arrange the extracted features in accordance with rules set by the respective classifiers. The classifiers are then configured to provide classified features to the personalized patient model 154.

The personalized patient model 154 is a model that builds associations between classified features in order to learn the meaning of the features and relationship between the features. The personalized patient model 154 is further trained using data collection and processing by feature extraction, classifiers, and associations in the model. The personalized patient model 154 will be specific to the patient. In some embodiments, the machine learning processor will generate multiple patient models, and each patient model can be instantiated for a specific patient, to best learn characteristics and identify patterns, trends and insights from the data sources. There are various types of machine learning algorithms that can be utilized to form and improve the personalized patient model 154. In some embodiments, the machine learning processor can utilize methods associated with supervised learning, unsupervised learning, and reinforced learning.

By way of example, the feedback learning data 164 can be utilized by a supervised learning process, wherein responses and feedbacks from either the patient or the doctor assist in the training process of the model to achieve higher levels of accuracy for the patient. Some embodiments of supervised learning include regression, decision tree, random forests, KNN, and logistic regression. In other embodiments, unsupervised learning utilizes methods to predict an estimate and outcome, e.g., by clustering data of different characteristics to identify patterns, trends, and insights automatically from the data. Some examples of unsupervised learning of algorithms include, without limitation, Apriori algorithm, K-means.

It is also possible to utilize reinforce learning, which a model teaches itself and is trained by continuously dealing with trial and error. The machine learning learns/AI from past experiences and tries to capture the best possible knowledge to make accurate decisions. An example of this type of processing, without limitation is a Markov Decision Process.

As shown, the personalized patient model 154 can therefore provide relevant information responsive to report request 160, which trigger a request to generate a summary report 116. The summer report 116 will access information that identify patterns trends and insights 114, wherein some of the information is gathered from the personalized patient model 154. In other embodiments, data used for the summary report 116 is output directly from one or more of the databases of the PCDA 100.

Some of the information, for example is simple identifying information of the patient, address information, contact information, and other static information. Some of information is more dynamic, and is generated in the form of insights or recommendations for the physician or care provider. As mentioned above, the summary report will advantageously include information that is gathered based on a synthesis of many types of data 104 that are gathered in collected 110, and process by filtering 112a using machine learning or simply gathering of data or metrics from simple filtering 112. The report request 160, as mentioned above, will trigger the request for the summary report.

The summary report can be in the form of data that is presented on one or more user interfaces of a device 182 of the patient, or device 184 of the care provider. Access to the PCDA 100 can be via a cloud interface 180, such as the Internet. The request can then be made to the information portal 160 of the PCDA 100. Data access processing 118 will gate and monitor access to the information, before being presented or exposed to care providers or the patient.

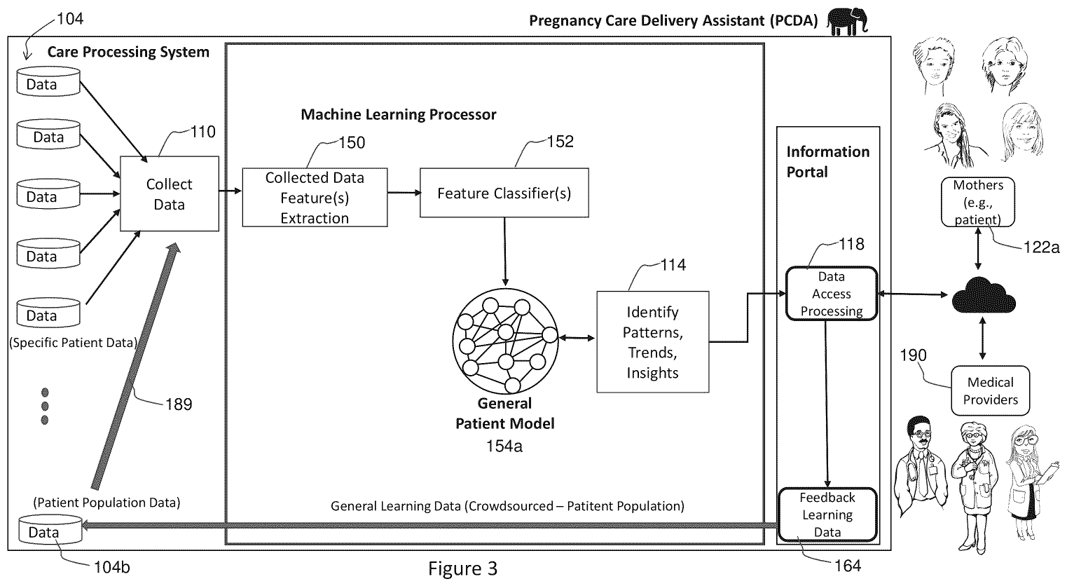

FIG. 3 illustrates an example of the PCDA 100, where processing done by the machine learning processor utilizes data gathered from the data sources 104, which include data source 104b, in accordance with one embodiment. Data source 104b, in one embodiment includes general learning data that is collected from many different patients 122a, to identify trends or patterns that can be utilized for the data collection 110. The general learning data received from feedback learning data 164, is useful for identifying trends that may be occurring in a specific characteristic of patient.

The characteristic can include age, prior symptoms, prior responses to care medication, prior complaints, prior responses, prior care programs, and the like. Collected data feature extraction 150 is processed on the collected data 110, which includes data 189 from the patient population data. Feature classifiers 152 are then used to label the features for consumption by the general patient model 154a. The general patient model 154a, in one embodiment, is generic and not specific to any one patient. However, the larger the data set that is ingested by the general patient model 154, the more effective the training can be for identifying patterns trends and insights 114.

As mentioned above, various types of machine learning algorithms can be utilized to generate the general patient model 154a, and requests to the general patient model 154a can be made by more than one data access processing 118 request. In some embodiments, when a guidance report includes information based on data processed and learning from the patient population, the report can identify that information is coming from the general population. This indicator in the report can be utilized to signal to the care provider as a potential care metric that can be utilized for delivering specific care to a current patient, utilizing the PCDA 100.

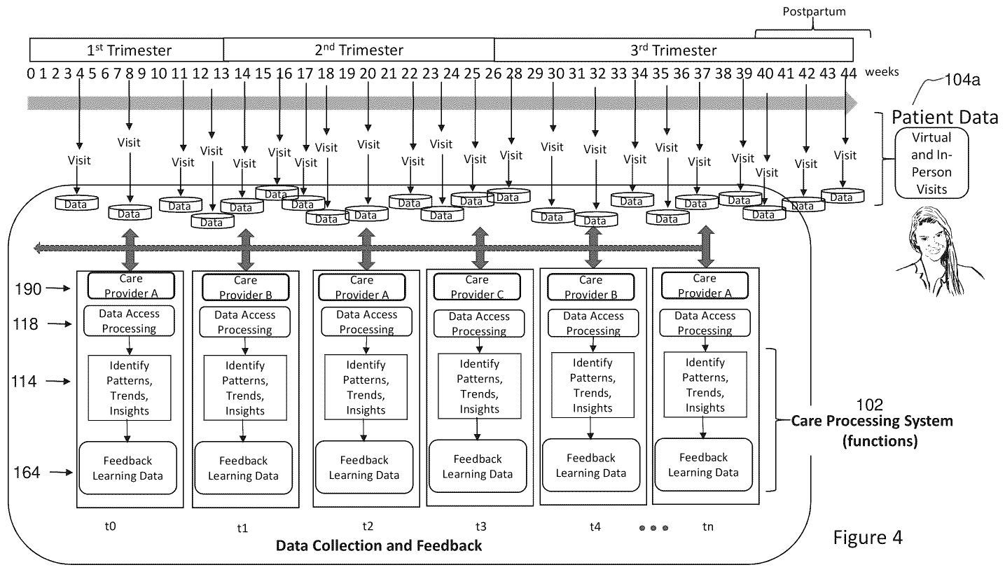

FIG. 4 illustrates an example of a pregnancy timeline, wherein a first trimester, second trimester, and a third trimester are illustrated broadly for discussion purposes. This illustration shows that patient data 104a can be gathered at any time throughout the pregnancy. This is shown by the illustration of multiple visits over time during the pregnancy cycle. The visits, as mentioned above, can be virtual visits utilizing computers, phones, videoconferences, remote monitoring devices, and the like. The visits can also be in-person meetings, where the care provider provide specific care routines. Typically, the patient data 104a will include a hybrid combination of virtual visits and in-person visits during the pregnancy cycle. Typically, the healthier the patient, the more effective virtual visits are in the system. Complicated pregnancy cases may require more in-person visits. In either case, data is generated during each of the visits.

This data can be gathered in many ways. The data can include patient vital signs, patient examination data, measurements, guidance data, administered care data, nutrition data, exercise data, past treatment data, future treatment recommendations, medications, etc. In one embodiment, as this data is being generated, the care processing system 102 will utilize functionality to capture the data and store it in one or more databases associated with the PCDA 100. In this illustration, it is shown that different care providers 190 can be providing care at different times of the pregnancy cycle. For purposes of illustration, care provider A, care provider B, and care provider C are shown as providers 190. This simple illustration shows that different care providers and sometimes the same care providers can provide care to the patient at different times.

In the simplest of examples, at t0 care provider A rendered care to the patient, at t1 care provider B rendered care to the patient, at t2 care provider A rendered care to the patient, at t3 care provider A rendered care to the patient, at t4 care provider B provided care to the patient, at to care provider A rendered care to the patient. If the patient were receiving care during a visit at week 25 (t3), the care provider C would typically not be aware of the specific care rendered by care provider A and B in prior visits. However, in accordance with one embodiment, the PCDA 100 could generate a report for the care provider C at time t3, and that report would take into consideration all of the data that occurred in previous visits.

Additionally, the report would include consideration of data mined from other data sources 104 as discussed above. Furthermore, using machine learning the report can include recommendations gleaned from insights and patterns identified by machine learning. Furthermore, using feedback learning data 164, one or more machine learning models can be continuously trained to provide more accurate insights, predictions, and recommendations that can be rendered in the report to the current care provider.

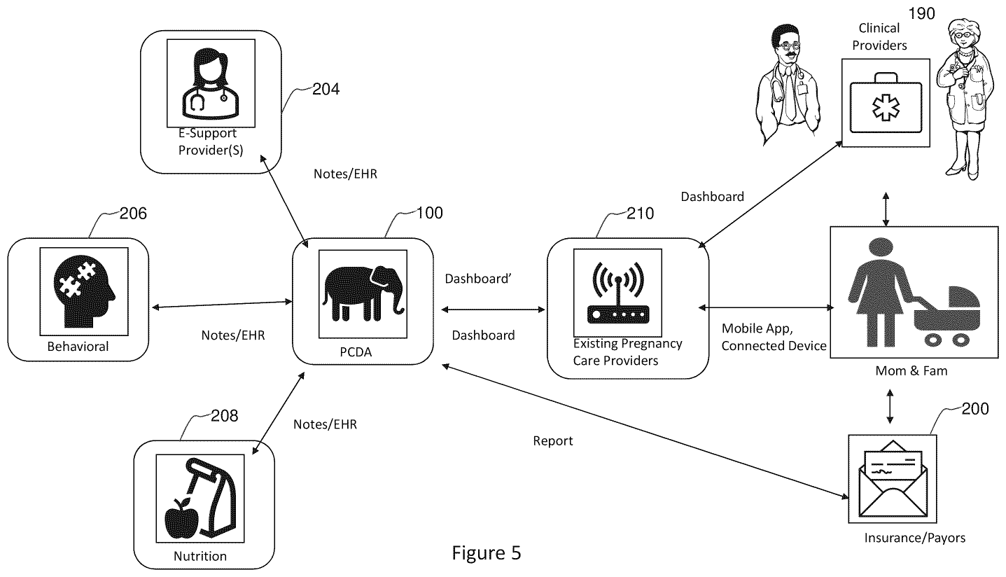

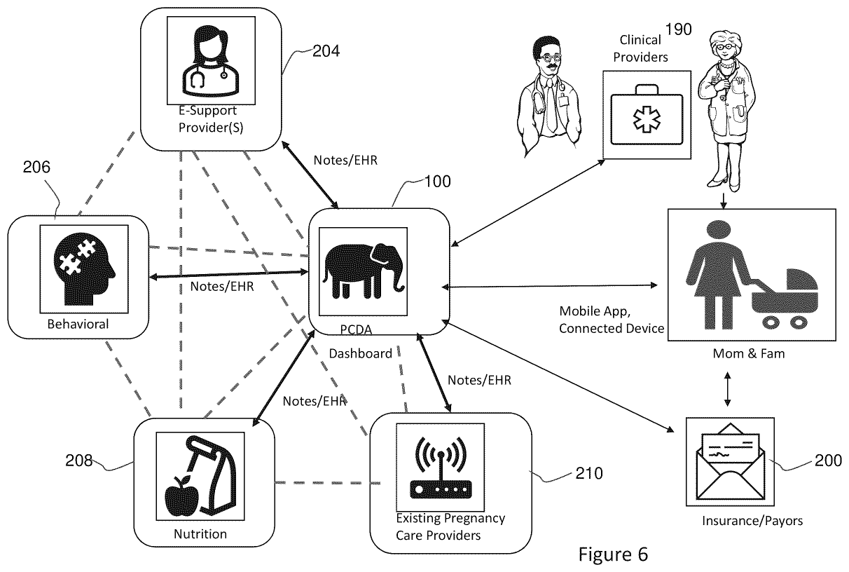

FIGS. 5 and 6 illustrate example data flow configurations when receiving data (e.g., data sources) from different pregnancy care ecosphere partners, in accordance with one embodiment. Ecosphere partners, by way of example, are entities that have systems and/or software for interfacing and providing care services to a mother. It is often the case that a mother may be receiving care from more than one provider, and such providers can be part of an ecosphere of partners. In some configurations, partners provide access to their systems so that data can be shared with the pregnancy care delivery assistant (PCDA), and/or modules of the care processing system. In one embodiment, the data may be accessed using one or more application programming interfaces (APIs) that are security option by the partners for use by the PCDA. In some configuration, the APIs communicate data in an encrypted format for security reasons. The data provided and obtained from such partners, in one embodiment represent the data sourced multiple entities and collected by the care processing system.

FIG. 5 shows PCDA 100 interfacing with other provider systems, such as the E-support providers 204, behavioral support providers 206, nutrition providers 208, and other existing front and pregnancy care providers 210. In this example, the existing pregnancy care providers 210 can provide the dashboard that utilizes information gathered by the PCDA 100. The dashboard provides the information portal for clinical providers 190, and insurance payers 200.

In one embodiment, the interfacing of these data sources with the PCDA enable data flow to and from the pregnancy care ecosphere partners. (Pregnancy Care Delivery Assistant). As shown in FIG. 6, the data availability for the pregnancy care “ecosphere” partners will also enhance their cross-ecosphere communication (dashed lines) on topics such as care for a specific patient, patient population, standard of care, etc. By way of example, having an E-support provider 204 technologically interfacing with the PCDA via data flow will provide both parties better pregnancy care situational awareness and hence facilitate better care for patients and better cross-ecosphere communication also in support of better patient care.

In one embodiment, the PCDA will provide for ancillary descriptive and predictive analysis that includes environmental and cost saving considerations associated with pregnancy care. Examples of environmental metrics are, without limitation to others, carbon emission reduction variables, patient distance from clinic or delivery unit, number of pregnancy care telehealth visits compared to standard all in-person visits, patient's primary vehicle and gas mileage, number of avoided labor and delivery or emergency department visits. Examples of cost savings variables are average cost of gas at time of avoided in-office visit, retained wages, retained PTO, retained childcare costs.

As shown in FIGS. 5 and 6, another user group that can receive information, reports and/or put from the PCDA is referred to a “payor” 200. In one embodiment, a payor could be health insurance, employers, and health systems. In one embodiment, potential insights this group would glean are billing code recommendations, population statistics such as reduced c-sections and associated costs with their patient population or comparative to another population, etc.

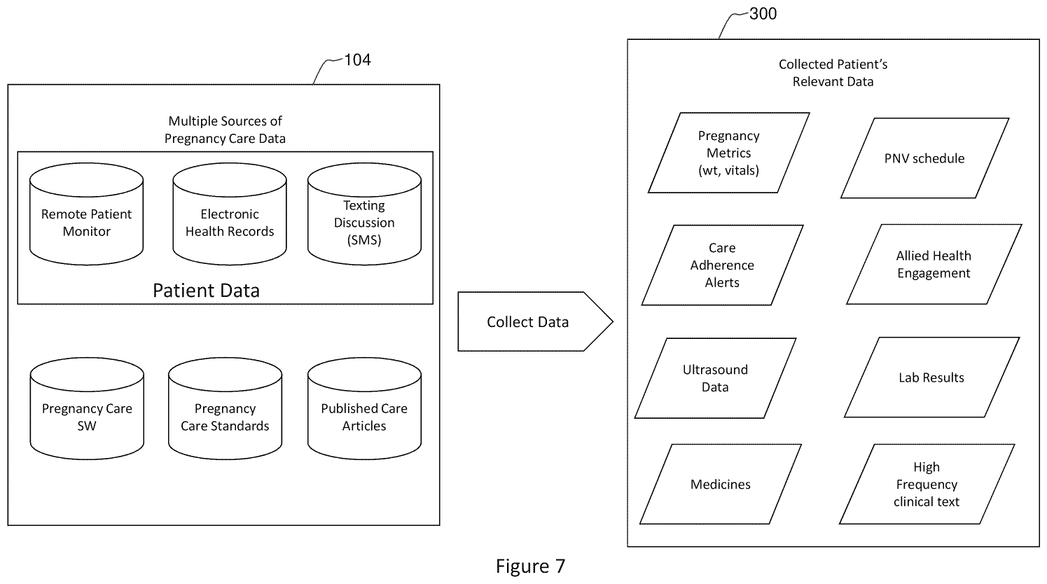

FIG. 7 illustrates an example of the multiple sources of pregnancy care data 104 and how collected data is then organized to define the patient's relevant collected data 300, in accordance with one embodiment. The patient data can include remote patient monitoring data, electronic health records, texting discussions, video call recordings, audio recordings, notes taken by care providers, vital signs captured, status reports, electronic health record systems (EHRs), laboratory facilities data, hospital EHRs (for patient emergency visits, admissions, deliveries, etc.), etc. It should be understood that the patient data that is collected as part of a data source 104 is only provided here by way of example, and additional data can be collected, stored, organized, filtered, and processed for generating one or more guidance reports. Other information can include pregnancy care software data. Pregnancy Care Software Data is a source of data originating from software that other allied health professionals or adjacent business/technology partners employ. An interface is established to collect data from these sources to enhance the holistic approach to overall pregnancy care that the PCDA is assisting.

Furthermore, the data source 104 can include pregnancy care standards data, and published care articles. In one embodiment, pregnancy care standards can be provided by multiple sources, and in some embodiments the multiple sources can be rated and/or prioritized. The collected data is then assembled or parsed to fill in one or more databases as mentioned above. In one embodiment, the collected data can include pregnancy metrics (the weight of the patient, the vitals of the patient, etc.), prenatal vitamin schedule (PNV), care adherence alerts, ultrasound data, medicines, allied health engagement, lab results, high-frequency clinical text, etc.

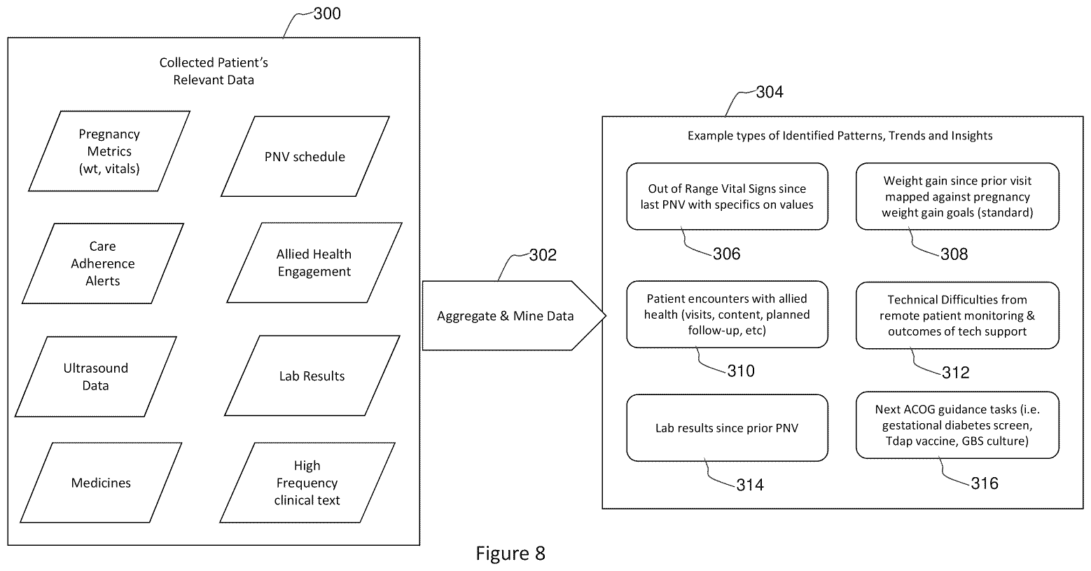

FIG. 8 illustrates an example where the collected patient's relevant data 300 can be aggregated and mined to generate relevant information that can be presented in a guidance report, in accordance with one embodiment. In this example, the aggregation and mining of data 302 can be performed by the filtering described above, including machine learning inputs for processing the patient's relevant collected data and other data that may be collected from other data sources as mentioned above.

By way of example, example types of identified patterns, trends and insights can be generated as shown in box 304. For instance, item 306 can be an output that signifies out of range vital signs since the last PNV and specifics on values. Item 308 can output information regarding weight gain since the prior visit mapped against pregnancy weight gain goals (e.g., standard). Item 310 can include information regarding the patient's encounters with allied health providers (e.g., visits, content, plan to follow up and the like.)

As mentioned above, allied health providers can be other care providers that may be rendering care to the patient. Some other health providers can provide care utilizing one or more Internet sites that facilitate information and or contact with health professionals of that provider. Those providers can collect information in their respective databases and the PCDA 100, in accordance with one embodiment, can source that information to provide additional insights when generating the guidance report 116. Item 312 may provide information regarding technical difficulties from remote patient monitoring equipment and/or outcomes from tech support. Item 314 can provide information regarding lab results since the prior PNV.

Item 316 can provide information regarding the next ACOG guidance tasks (i.e., gestational diabetes screen, Tdap vaccine, GBS culture, etc.). As can be appreciated, the identified patterns, trends, and insights may be gathered from multiple sources, and rendering of this information can include filtering such that only pertinent information for the patient at the specific current time of the report is rendered. Additionally, information that is not relevant or not related to the patient may be eliminated. Additionally, information that is predicted to be important based on a population of other patients can also be rendered in the reports to assist the care provider.

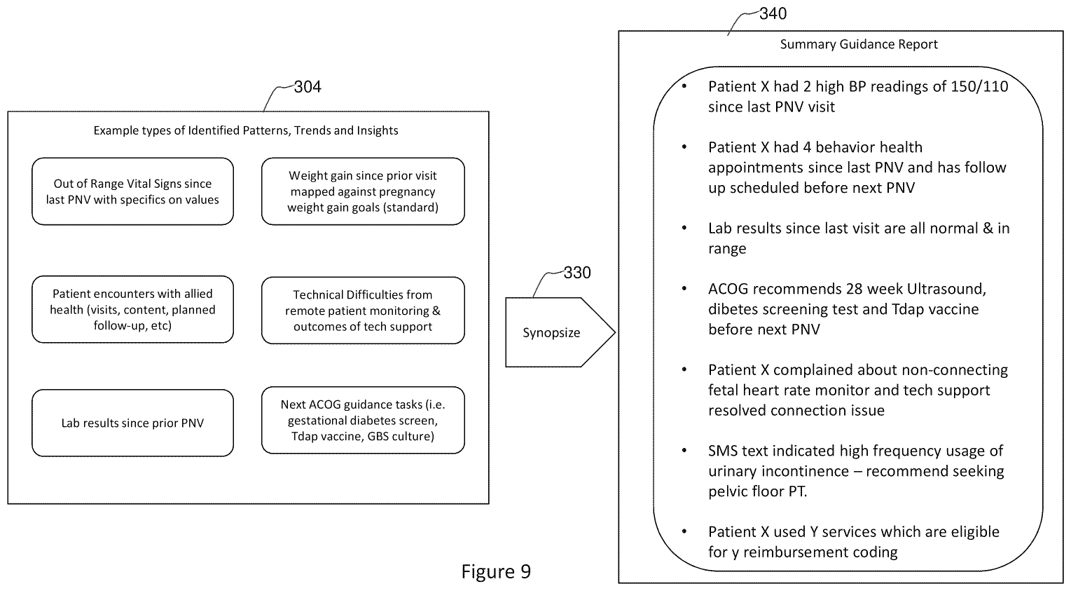

FIG. 9 illustrates an example of utilizing the types of patterns, trends and insights 304 in order to generate a summary guidance report 340, in accordance with one embodiment. As illustrated, the generated information in the guidance report can be in the form of statements that are most relevant to a current visit of the patient, looking back at prior visits and data collected, and all other relevant sources that are analyzed during the data collection.

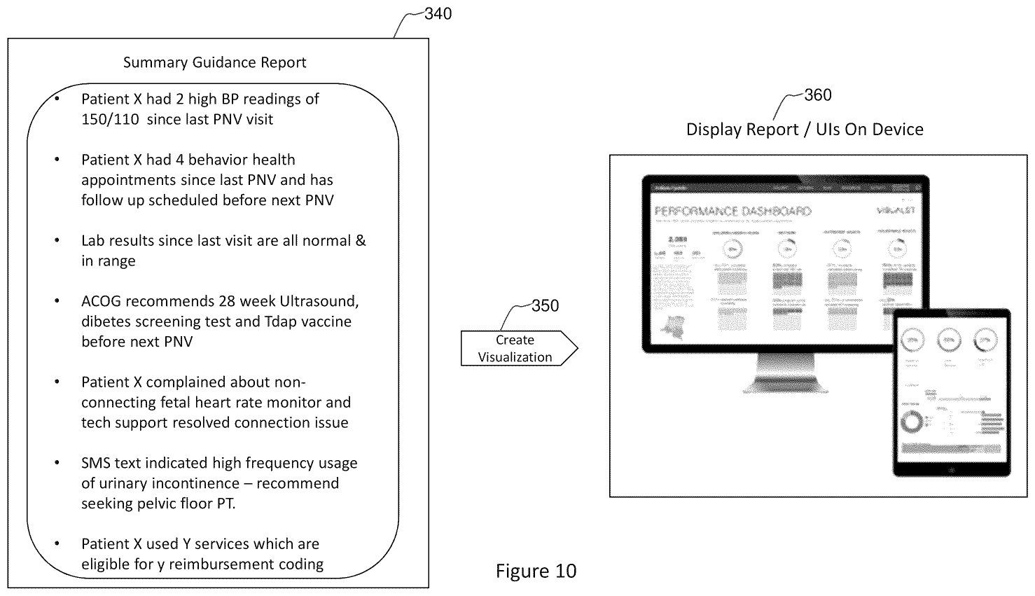

FIG. 10 illustrates an example of the visualization 350 that can be created from the data in the summary guidance report 340. As shown, the report can be displayed 360 in multiple UIs and multiple devices, depending on the information being delivered. It should be understood that the summary guidance report information can be displayed in many forms, including graphical user interfaces, tables, graphs, pie charts, interactive screens, recommendation screens, recommended treatments, and other helpful information for the care provider and/or the patient who may be accessing the PCDA 100 using a device.

In some embodiments, data elements that can be displayed in dashboards as a result of PCDA analysis include, but are not limited to:

Patient's Contact and Identification.
Patient name
Patient date of birth
Patient phone number
Patient emergency contact
Patient address
Patient e-mail address

Patient's basic pregnancy data.
Due Date (Gestational Age)
Program enrollment date
Practice name and location (primary OB care team)
Intake information and dating
Clinical care data and software access

Ecosphere sources in use (other PCDA connected pregnancy care allied health providers being used such as doula, nutrition, behavioral health, pelvic floor support, etc.).
Date and outcome of last encounter
Confirmed communication to primary OB care team via Electronic Health Record integration
Ability for primary OB care team to communicate with ecosphere partners

Patient Adherence.
Last vital sign self-assessment
Current prenatal visit metrics
Next prenatal visit (Telehealth/in-office)

Patient Clinical Support.
Clinical care dashboard access to primary OB care team and PCDA (e.g., implementing an e-Lōvu Health service) clinical support team

Green Calculator (environmental and financial savings).
Carbon emission used or saved
Personal cost savings (gas, electricity, childcare, paid time off (PTO) . . . ).

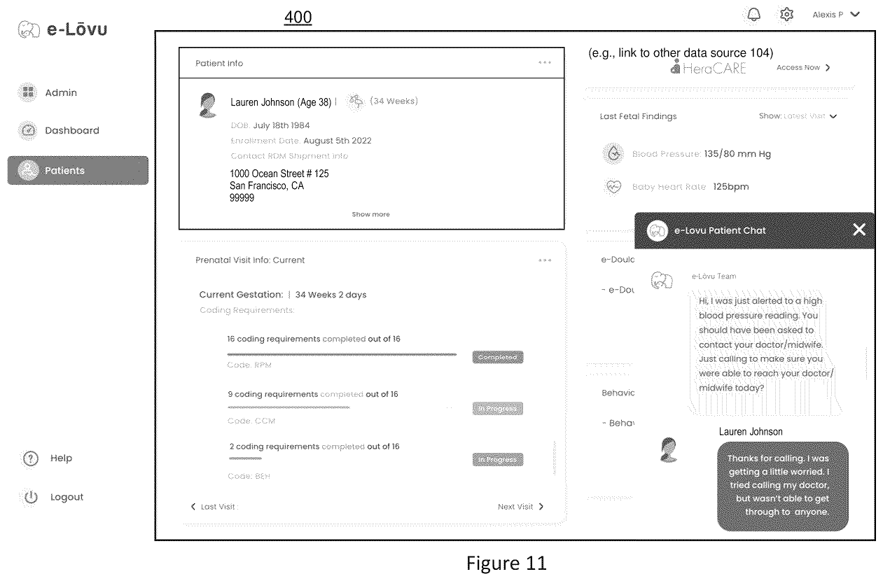

FIG. 11 is a screenshot 400 example showing an individual patient dashboard that is displayed as a result of the PCDA output, in accordance with one embodiment. In one embodiment, this is output from the PCDA information portal 106, responsive to a request received by the PCDA over a network connection. Examples of data displayed within this view is patient identification information, on-going SMS (e.g., text message) discussions between patient and care team, ecosphere partner engagement metrics, data and guidance from last pre-natal visit, data and guidance for current pre-natal visit, data and guidance for next pre-natal visit. This information output can also include other timely pregnancy care data, such as current ultrasound readings, lab results, vaccinations, etc.

In one embodiment, the request 160 made via the information portal 106 is processed in substantial real-time, enabling the access of data, filtering of data, and generation of guidance data for a current time of when the request was made. In this configuration, the guidance data will utilize historical data that occurred before the current visit and request, including access to public and/or private pregnancy care best practices, pregnancy care standards and/or updates to standards, and information identified and consumed from published care articles.

In another embodiment, the request 160 made via the information portal 106 is processed responsive to the request 160, but some data presented in the guidance data is pre-computed and cached in storage of the care processing system 102. As mentioned above, the PCDA is, in one embodiment, processed by one or more servers that have access to storage. The processing logic may be executed by processors of the servers, and such servers may be programmed to execute routines to filter data at different times and cache results. The cloud system for executing the PCDA can be Amazon™ Web Services (AWS™). Although any other cloud system may be used for processing operations of the PCDA, and other front-end software systems may be interfaced for providing user facing graphical user interfaces on any access device, AWS is provided as one example only. In AWS, the compute can be executed by an Amazon EC2 virtual server in the cloud. This configuration will enable scaling of compute capacity on demand. In some embodiments, elastic container services may be used to enable secure and container execution for sensitive data processing.

In still another embodiment, Google™ Cloud systems may also be used to process compute engines used by the PCDA in a cloud configuration for performance and scalability. Data is further storable and scalable using Google Storage, which may use object storage, and persistent disk block storage. In some embodiments, databases used for storing data mined from the disparate data sources. The databases can include, e.g., MySQL, PostgreSQL, Cloud SQL, and other custom and/or third-party databases. In some configurations, when data is obtained from the care provider ecosystem, the data may be accessed using one or more APIs, and collected data can be identified, sorted and stored in a PCDA database.

The PCDA database, will in one embodiment, contain custom fields that are specific for care provider data and useful for efficient processing by one or more rule engines. The one or more rule engines are executed in the cloud and enable the filtering used by the care processing system 102. In one embodiment, output from the machine learning processor can be used to populate data into one or more of the PCDA databases and associated to specific patients or can be tagged as applicable to a population of patients.

The cached results, therefore, enable more efficient generation of guidance data when requests are made. To maintain an up-to-date cache, the caching can be performed on a predefined schedule that ensures data is current and accurate for requests that may be received at any time over the Internet, be it from a care provider or the patient.

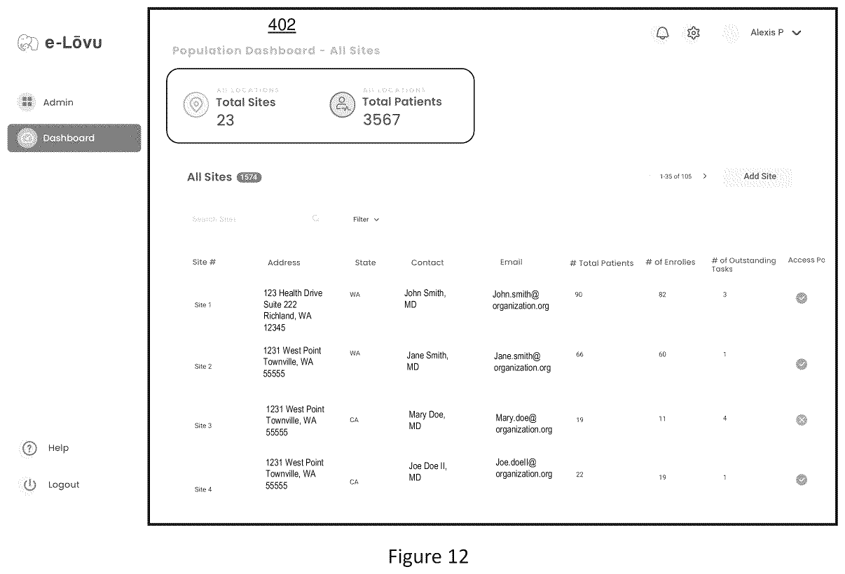

FIG. 12 is a screenshot 402 example showing an all-site population dashboard that is displayed as a result of the PCDA output, in accordance with one embodiment. By way of example, this is output from the PCDA information portal 106. Examples of data displayed within this view are total number of sites, practices and patients, site and practice contact information, number of site's outstanding tasks, number of new enrolled patients, etc.

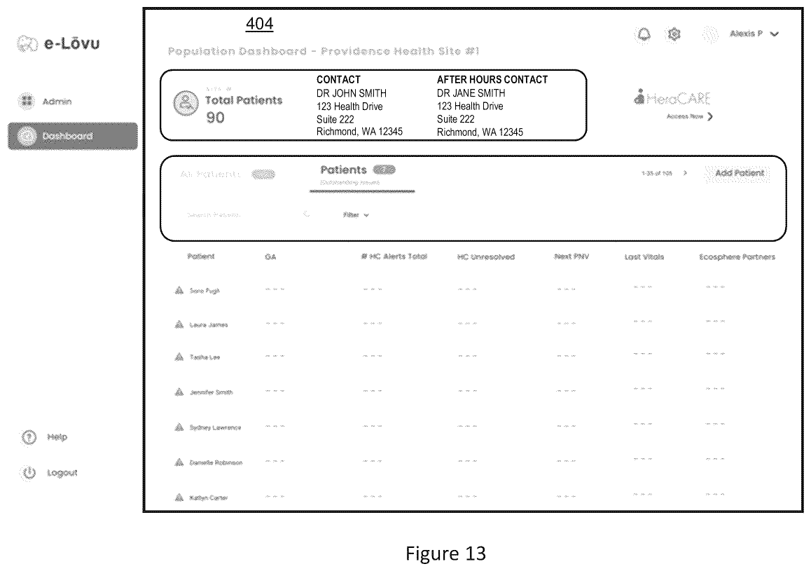

FIG. 13 is a screenshot 404 example showing the specific site population dashboard that is displayed as a result of the PCDA output, in accordance with one embodiment. This is output from the PCDA information portal 106. This example view provides a site-specific patient data summary Examples include highlighting patients with outstanding issues (e.g., unresolved healthcare alerts), patient's gestational ages, patient's pre-natal visit timing, patient's contact information and identification ecosphere partner usage, as well as a button to double click for access to individual patient care dashboard and clinical care partners. As mentioned above, this data is organized in one or more tables of a database or databases used by processing of the PCDA, and when accessed can be used by the information portal 106 for presentation on one or more graphical user interfaces, screens, displays and/or devices that have access over a networked connection, wireless or wired and/or over the Internet using any device.

In some configurations, patient data that may be used anonymously for generating inferences and ML/AI processing may be further protected. By way of example, in some embodiments, patients may be provided with some ownership of their personal medical data. Ownership may be protected using blockchain technology, which allows patients an ability to also earn a small amount of income whenever their de-identified data is used by third parties for the purpose of improving healthcare delivery. It should be understood that the data, when used, is used anonymously. Nevertheless, patients may in some configurations be provided with an option to assist in improving healthcare delivery, while also receiving some income when the use occurs.

Embodiments of the present invention may be practiced with various computer system configurations including servers, cloud systems, hand-held devices, microprocessor systems, microprocessor-based or programmable consumer electronics, minicomputers, mainframe computers and the like. The invention can also be practiced in distributed computing environments where tasks are performed by remote processing devices that are linked through a wire-based or wireless network.

With the above embodiments in mind, it should be understood that the invention could employ various computer-implemented operations involving data stored in computer systems. These operations are those requiring physical manipulation of physical quantities. Usually, though not necessarily, these quantities take the form of electrical or magnetic signals capable of being stored, transferred, combined, compared and otherwise manipulated.

Any of the operations described herein that form part of the invention are useful machine operations. The invention also relates to a device or an apparatus for performing these operations. The apparatus can be specially constructed for the required purpose, or the apparatus can be a general-purpose computer selectively activated or configured by a computer program stored in the computer or storage in cloud systems. In particular, various general-purpose machines can be used with computer programs written in accordance with the teachings herein, or it may be more convenient to construct a more specialized apparatus to perform the required operations.

The invention can also be embodied as computer readable code on a computer readable medium. The computer readable medium is any data storage device that can store data, which can thereafter be read by a computer system. The computer readable medium can also be distributed over a network-coupled computer system so that the computer readable code is stored and executed in a distributed fashion. The method operations were described in a specific order, it should be understood that other housekeeping operations may be performed in between operations, or operations may be adjusted so that they occur at slightly different times, or may be distributed in a system which allows the occurrence of the processing operations at various intervals associated with the processing, as long as the processing of the overlay operations are performed in the desired way.

Although the foregoing embodiments have been described in some detail for purposes of clarity of understanding, it will be apparent that certain changes and modifications can be practiced within the scope of the appended claims. Accordingly, the present embodiments are to be considered as illustrative and not restrictive, and the embodiments are not to be limited to the details given herein, but may be modified within the scope and equivalents of the appended claims.

What is claimed is:

1. A method for processing pregnancy care data for presentation via an information portal, comprising:
receiving a plurality of data streams from a plurality of data sources that are interfaced with a care processing system;
filtering data from the plurality of data streams, the filtering is configured to identify relevant data to a patient and a current status of the patient;
identifying at least one pattern from the relevant data;
generating a guidance report regarding the patient, the guidance report is configured to be generated responsive to a request received from a care provider for a visit or the patient, wherein the guidance report synthesizes said relevant data from one or more prior visits with the care provider or another care provider that provided care before the current status of the patient.

2. The method of claim 1, wherein said plurality of data sources include data related or collected from the patient and data related to a patient population having similar care characteristics to the patient, wherein the similar care characteristics are at least partially used to assist in identifying said at least one pattern.

3. The method of claim 1, wherein the filtering data further includes using a machine learning processor that is configured to parse collected data from each of the plurality of data sources to identify features for related types of data, the features are then processed by classifiers that are specific to the types of data, wherein output of the classifiers are processed to generate a patient model.

4. The method of claim 1, wherein the guidance report, when synthesized, provides metrics recorded in said one or more prior visits, the metrics are present in a descriptive format that is presented in one or more user interfaces, the one or more user interfaces are for display on a user device having access to the care processing system via an information portal.

5. The method of claim 1, wherein the identified at least one pattern from the relevant data is identified using a machine learning processor that extracts feature data from the plurality of data sources and labels features for classification and processing by model, the model is used to identify said at least one pattern.

6. The method of claim 5, wherein the plurality of data sources includes patient population data and associated feedback learning data to train said model.

7. The method of claim 1, wherein a machine learning model is used to identify said at least one pattern.

8. The method of claim 1, wherein one or more rules engines are used to identify said at least one pattern.

9. The method of claim 8, wherein the rules engines are programmable to identify specific data from said plurality of data sources and apply logic to represent said specific data in a descriptive format that describes an aspect of prior care of the patient.

10. The method of claim 1, wherein said relevant data is identified from one or more prior visits with the care provider or another care provider that provided care before the current status of the patient, and said relevant data is processed by at least one rules engine that identifies a significance of the relevant data in relation to the patient.

11. A method for processing pregnancy care data for presentation via an information portal, comprising:
accessing a plurality of data sources having care data related to a patient;
filtering data from the plurality of data sources, the filtering is configured to identify relevant data to the patient responsive to a request for a guidance report for pregnancy care of the patient via the information portal;
identifying at least one pattern from the relevant data;
generating the guidance report regarding the patient, wherein the guidance report includes relevant data from one or more prior visits with a care provider.

12. The method of claim 11, wherein said plurality of data sources include data related or collected from the patient and data related to a patient population having similar care characteristics to the patient, wherein the similar characteristics are at least partially used to assist in identifying said at least one pattern.

13. The method of claim 11, wherein the filtering data further includes using a machine learning processor that is configured to parse collected data from each of the plurality of data sources to identify features for related types of data, the features are processed by classifiers that are specific to the types of data, wherein output of the classifiers are processed to generate a patient model, the patient model is trained over time using feedback from the care provider or the patient.

14. The method of claim 1, wherein the guidance report, when generated, provides metrics recorded in said one or more prior visits, the metrics are present in a descriptive format that is presented in one or more user interfaces, the one or more user interfaces are for display on a user device having access to the care processing system via the information portal.

15. The method of claim 1, wherein the identified at least one pattern from the relevant data is identified using a machine learning processor that extracts feature data from the plurality of data sources and labels features for classification and processing by model, the model is used to identify said at least one pattern or generate an insight to be presented in the guidance report.

16. The method of claim 15, wherein the plurality of data sources includes patient population data and associated feedback learning data to train said model, including data sourced from other online care providers via one or more Application Programming Interfaces (APIs).

17. The method of claim 11, wherein a machine learning model is used to identify said at least one pattern.

18. The method of claim 11, wherein one or more rules engines are used to identify said at least one pattern.

19. The method of claim 18, wherein the rules engines are programmable to identify specific data from said plurality of data sources and apply logic to represent said specific data in a descriptive format that describes an aspect of prior care of the patient.
